(19) United States
(12) Patent Application Publication
(10) Pub. No.: US 2021/0204877 A1
(43) Pub. Date: Jul. 8, 2021

(54) TEXTILE COMPUTING PLATFORM IN SLEEVE FORM

(71) Applicant: MYANT INC., Toronto (CA)

(72) Inventors: Milad ALIZADEH-MEGHRAZI, Toronto (CA); Adrian STRAKA, Toronto (CA); Godfried EDELMAN, Toronto (CA); John PERSIC, Toronto (CA); Kilian O'DONOGHUE, Toronto (CA); Tony CHAHINE, Toronto (CA)

(21) Appl. No.: 17/057,031

(22) PCT Filed: May 22, 2019
(86) PCT No.: PCT/CA2019/050696

§ 371 Date: Nov. 19, 2020

Related U.S. Application Data
(60) Provisional application No. 62/674,694, filed on May 22, 2018.

(51) Int. Cl. A61B 5/00; A61B 5/0537; A61B 5/08; A61B 5/0533; A61B 5/318; A61B 5/389; A61B 5/369; A61B 5/01; A61B 5/1468

(52) U.S. Cl. CPC A61B 5/6804; A61B 5/398; A61B 5/08; A61B 5/0533; A61B 5/318; A61B 5/389; A61B 5/369; A61B 5/01; A61B 5/7455; A61B 5/4266; A61B 5/1468; A61B 5/4839; A61B 2562/0247; A61B 2562/0261; A61B 5/0537

(57) ABSTRACT

A textile-based computing platform for wearing by a wearer on both sides of a joint of a body of the wearer, the platform comprising: a textile body shaped as a sleeve including a first zone for positioning adjacent to the joint, a second zone opposite the first zone for positioned on another side of the joint, and an intermediate zone for positioning over the joint; a fabric sensor incorporated into a textile layer making up the textile body, a fabric actuator incorporated into the textile layer making up the textile body, an electrical connector mounted on the textile body for connecting to a controller computing device; an electronic circuit coupling the electrical connector to the fabric sensor and the fabric actuator, the circuit electrically conductive threads incorporated into the textile layer.

### CROSS-REFERENCE TO RELATED APPLICATIONS

This application claims the benefits of U.S. Provisional Patent Application Ser. No. 62/674,694, filed on May 22, 2018; the entire contents of which are hereby incorporated by reference herein.

### BACKGROUND

A central need for garment wearers during certain activities is to become able to sense, what the body is doing: which muscles are flexed? Are the joints properly flex/angled? The ability for the garment wearer to ascertain biometric and orientation information about selected parts of the body becomes even more pronounced during physiotherapy or other recuperative activities. Accordingly, needs in the areas of medicine and rehabilitation or physiotherapy is for tracking of movements of specific body parts, in particular for range of motion for recuperation therapies, as well as for swelling/enlargement of body parts due to disease or other medical conditions. Again, historical tracking of body movement is needed to facilitate treatment in these areas, however current movement sensing clothing is cumbersome at best. For example, placement of particular sensors (e.g. stretch sensors) adjacent to specified body parts can be difficult due to repeatable positioning difficulties of the sensors, as well as maintaining of the sensors in position during the body movements being tracked/monitored.

### SUMMARY

A first aspect provided is a textile-based computing platform for wearing by a wearer on both sides of a joint of a body of the wearer, the platform comprising: a textile body shaped as a sleeve including a first zone for positioning adjacent to the joint, a second zone opposite the first zone for positioned on another side of the joint, and an intermediate zone for positioning over the joint; a fabric sensor incorporated into a textile layer making up the textile body, the fabric sensor having one or more electrically conductive sensor threads incorporated into the textile layer by at least one of knitting or weaving with other threads making up the textile layer; a fabric actuator incorporated into the textile layer making up the textile body, the fabric actuator having one or more electrically conductive actuator threads incorporated into the textile layer by at least one of knitting or weaving with the other threads making up the textile layer; an electrical connector mounted on the textile body for connecting to a controller computing device; an electronic circuit coupling the electrical connector to the fabric sensor and the fabric actuator, by way of circuit electrically conductive threads connected to the one or more electrically conductive actuator threads and the one or more electrically conductive sensor threads, the circuit electrically conductive threads incorporated into the textile layer by at least one of knitting or weaving with the other threads making up the textile layer; wherein the controller computing device when connected to the electrical connector bidirectionally communicates electrical signals via the electronic circuit with respect to at least one of the fabric sensor and the fabric actuator.

The textile-based computing platform can be in one or more form factors applicable to a joint, such as but not limited to a knee joint, an elbow joint, and an ankle joint.

A second aspect provided is a textile-based computing platform in the shape of an eye band.

A third aspect provided is a textile-based computing platform in the shape of a head band.

A fourth aspect provided is a textile-based computing platform incorporated into a garment for wearing on a torso or midsection of a wearer.

A fifth aspect provided is a textile-based computing platform in the shape of a covering for a head of a wearer.

### BRIEF DESCRIPTION OF THE DRAWINGS

The non-limiting embodiments may be more fully appreciated by reference to the following detailed description of the non-limiting embodiments when taken in conjunction with the accompanying drawings, by example only, in which:

FIG. 1 provides examples of locations on a wearer's body for positioning of a textile-based computing platform, including locations overlapping a selected body joint;

FIG. 2 is an embodiment of the textile-based computing platform of FIG. 1 in a sleeve form factor;

FIG. 3 is a further embodiment of the textile-based computing platform of FIG. 2 as a perspective rear view;

FIG. 4 shows a front view of the textile-based computing platform of FIG. 3;

FIG. 5 shows a side view of the textile-based computing platform of FIG. 3;

FIG. 6 shows a further opposite side view of the textile-based computing platform of FIG. 3;

FIG. 7 shows a perspective front view of the textile-based computing platform of FIG. 3;

FIG. 8 shows an example sensor/actuator application of the textile-based computing platform of FIG. 3;

FIG. 9 shows a further example sensor/actuator application of the textile-based computing platform of FIG. 3;

FIG. 10 shows a further example sensor/actuator application of the textile-based computing platform of FIG. 3;

FIG. 11 shows a further example sensor/actuator application of the textile-based computing platform of FIG. 3;

FIG. 12 shows an opposite side view of the textile-based computing platform of FIG. 11;

FIG. 13 shows a further example sensor/actuator application of the textile-based computing platform of FIG. 2;

FIG. 14 shows a further example sensor/actuator application of the textile-based computing platform of FIG. 2;

FIG. 15 shows a further example sensor/actuator application of the textile-based computing platform of FIG. 2;

FIG. 16 shows a further example sensor/actuator application of the textile-based computing platform of FIG. 2;

FIG. 17 shows a further example of the the textile-based computing platform of FIG. 1;

FIG. 18 shows a further example of the the textile-based computing platform of FIG. 1;

FIG. 19 shows an example textile forming structure as knitting including one or more sensors/actuators of the textile-based computing platform of FIG. 1; and

FIG. 20 shows a further example textile forming structure as weaving including one or more sensors/actuators of the textile-based computing platform of FIG. 1;

### DETAILED DESCRIPTION OF THE EMBODIMENTS

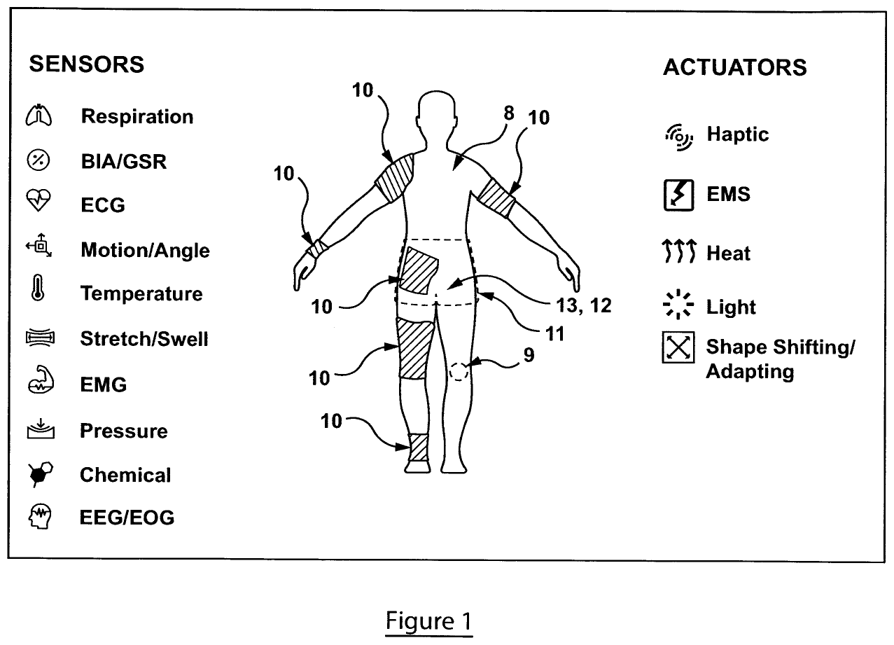

Referring to FIG. 1, shown is a body 8 of a wearer for wearing one or more textile based computing platforms 10 positioned about one or more joints 9 (e.g. knee, ankle, elbow, wrist, hip, shoulder, neck, etc.) of the body 8. For sake of simplicity, textile based computing platforms 10 can also be referred to textile computing platforms 10. For example, the textile computing platforms 10 can also be referred to as a wrist sleeve 10, a knee sleeve 10, a shoulder sleeve 10, an ankle sleeve 10, a hip sleeve 10, a neck sleeve 10, etc. It is also recognized that the textile computing platform 10 can be incorporated as part of a larger garment 11 (e.g. a pair of briefs 11 as shown in ghosted view for demonstration purposes only). It is recognized that the garment 11 could also be a shirt, pants, a body suit, as desired. As such, the fabric/textile body 13 of the garment 11 can be used to position the textile computing platform 10 for those areas of the body 8 where a sleeve based form factor of the textile computing platform 10 would be awkward for the wearer.

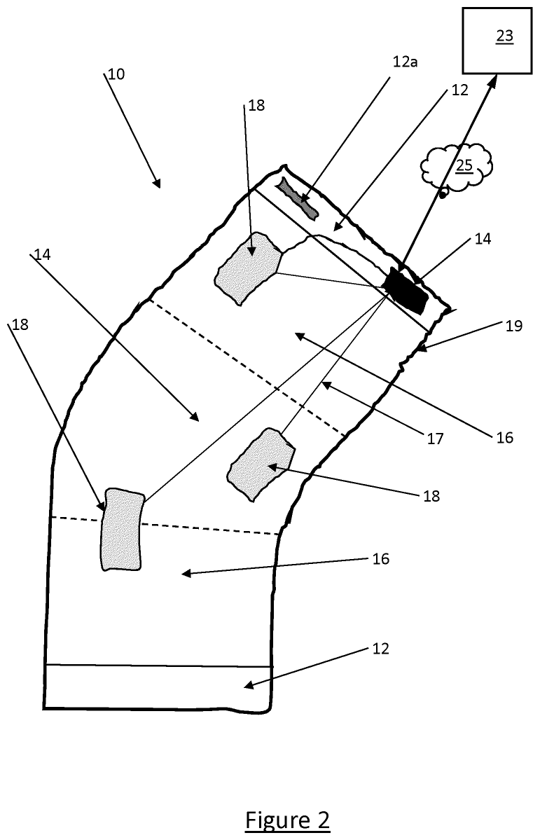

Referring to FIGS. 1 and 2, positioning of the textile computing platform 10 on the body 8 of the wearer is preferably done on and to one or both sides of the joint 9. Retaining positioning of the textile computing platform 10 about the joint 9 during continued motion (e.g. flexion) of the joint 9 can be provided by one or more bands 12 and/or the body 13 of the garment 11 (when used). The body 13 and the bands 12 can be generically referred to as position retainers 12, incorporating one or more compressive textile sections 12a for helping to maintain contact of the position retainers 12 with the surface of the body 8. For example, the compressive textile sections 12a can be knitted ribs, fabric containing elastic fibres, elastic bands, etc.

Referring again to FIG. 2, the textile computing platform 10 includes a textile/fabric body 19 (e.g. woven and/or knitted, seamed and/or seamless, as desired) that can have a plurality of zones 14,16. The zone(s) 16 can be positioned to one or either side of the zone 14, recognizing that the zone 14 is meant to be positioned and retained (i.e. by the position retainers 12) over the joint 9. The textile computing platform 10 can also have a controller 14 for sending/receiving signals to one or more sensors/actuators 18 distributed about the body 19. The shape of the sensors/actuators 18 can be elongate (e.g. as a strip extending in a preferred direction) or can extend as a patch in a plurality of directions (e.g. extend side to side and end to end). The signals are transmitted between the sensors/actuators 18 and the controller 14 via one or more electronic circuits 17 connecting the controller 14 to each of the sensors/actuators 18. It is also recognized that the electronic circuits 17 can also be between individual sensors/actuators 18, as desired. As further described below, the sensors/actuators 18 can be textile based, i.e. incorporated via knitting/weaving as part of the fabric layer of the body 19, formed as a plurality of threads of electrically conductive and optionally non-conductive properties). Further, the electronic circuits 17 (e.g. electrically conductive threads) can also be incorporated (e.g. knitting/weaving) into the fabric layer of the body 19. The controller 14, further described below, can include a network interface (e.g. wireless or wired) for communicating with a computing device 23 (e.g. smart phone, tablet, laptop, desktop, etc.) via a network 25.

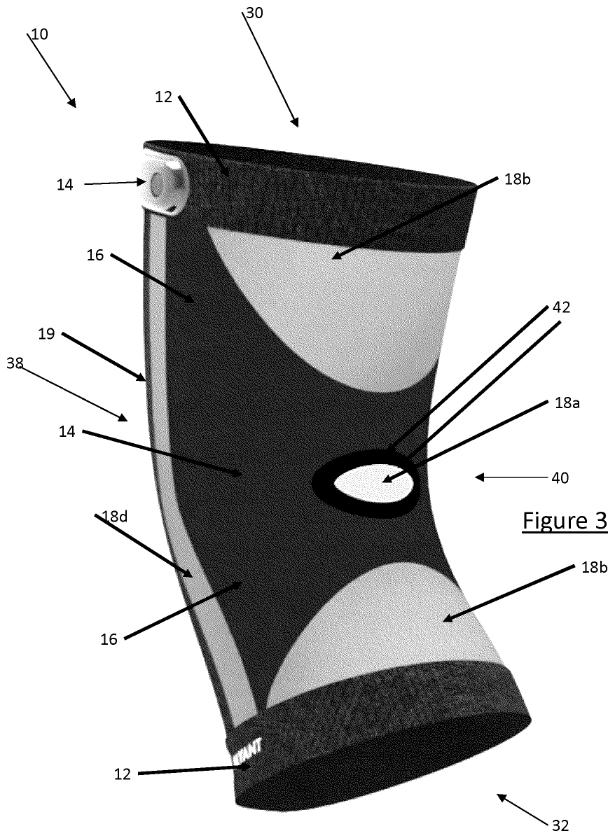

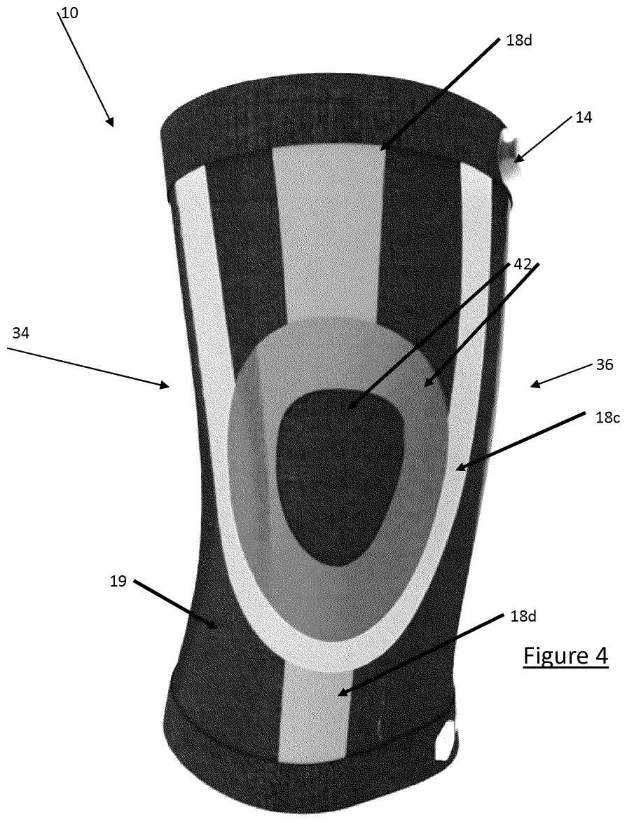

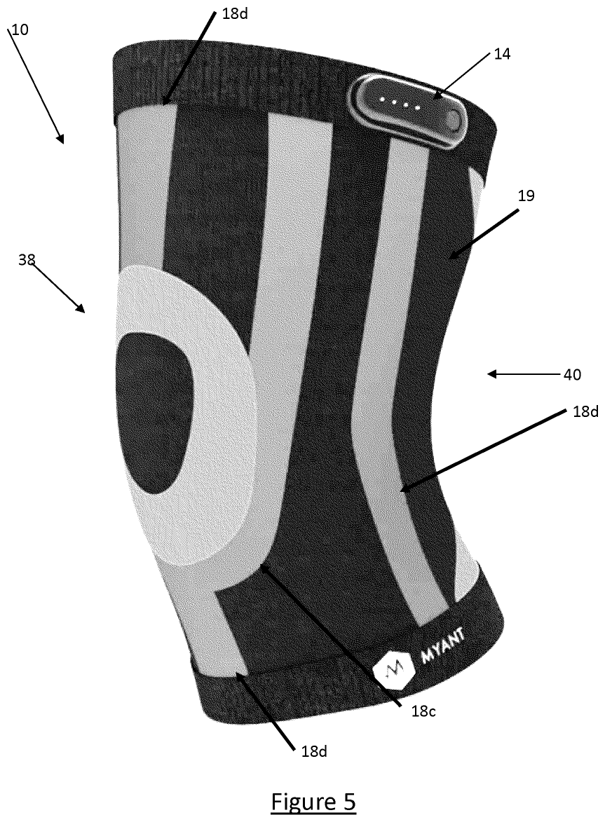

Referring again to FIG. 2, the sensors/actuators 18 can be positioned completely within a respective zone 14, 16 or can straddle two or more adjacent zones 14,16, as desired. Specific examples of sensor/actuator 18 type and zone 14,16 positioning are presented further below. One embodiment of the textile computing platform 10, see FIG. 3, is provided as a sleeve 10 having a pair of position retainers 12 (e.g. bands) at either end 30,32 the body 19 having a pair of respective zones 16 adjacent to the position retainers 12 with an intermediate zone 14 positioned between the pair of zones 16. It is recognized that the zone 14 is shaped so as to be positionable over the joint 9 (see FIG. 1), while the zones 16 are shaped to be positionable to either side of the joint 9. For example, in the case of an elbow or knee sleeve 10, the body 19 fabric of one of the zones 16 (adjacent end 30) could be of a greater diameter that the other zone 16 (adjacent end 32), in order to account for limb thickness differences to either side of the knee/elbow joint 9. Similarly, the retainer portion 12 (e.g. band 12) adjacent to the zone 16 (adjacent end 30) of greater diameter would also be of greater diameter than the other position retainer 12 (adjacent end 32) adjacent to the relatively smaller diameter zone 16. Referring to FIGS. 4 and 5, the body of the sleeve 10 also has a pair of sides 34,36 extending between the ends 30,32, as well as second pair of sides 38,40 also extending between the ends 30,32, such that the sides 34,36,38,40 comprise the sleeve for surrounding the body 8 of the wearer about the joint 9. It is recognized that side 38 is positioned between opposed sides 32,34 and side 40 is also positioned between opposed side 32,34, such that sides 38,40 are opposed to one another.

Referring again to FIGS. 3,4, the sleeve 10 can have one or more position indicators 42 (e.g. portion of the body 19 fabric) for indicating proper positioning of the body 19 with respect to the joint 9. For example, it is recognized that different types of the sensors/actuators 18 can have particular locations on/in the body 19 and therefore each of the sides 34,36,38,40 are meant to be oriented about the joint 9 as one side, an opposite side, a front and a back. For example, the position indicator 42 can be of a different/distinctive fabric color or texture or geometric shape compared to the rest of the fabric of the body 19, so as to orient the wearer as to how best to position the sleeve 10 with respect to the joint 9. For example, as shown in FIG. 4, the indicator 42 can be one or more circles indicating the apex position of the joint 9, as well as front verses back of the joint 9. In the present embodiment, the larger circle indicator 42 (shown in FIG. 4) is intended to be positioned on the front of the joint 9 (e.g. over the knee on the front of the wearer's leg 8), while the smaller circle indicator 42 (shown in FIG. 3) is intended to be positioned on the rear of the joint 9 (e.g. behind the knee on the rear of the wearer's leg 8).

Referring to FIGS. 3,4,5,6,7, the embodiment shown for the textile computing platform 10 has a plurality of sensors/actuators 18. For example, positioned on opposed sides 38,40 are electro-muscular stimulators (i.e. actuators) 18a for applying an electrical stimulation signal (e.g. a shock) to the skin and underlying muscles of the wearer adjacent to the electro-muscular stimulators 18a (e.g. for facilitating pain relief). It is recognized that the electro-muscular stimulators 18a are positioned in the intermediate zone 14, such that one or both of the electro-muscular stimulators 18a can be present in the zone 14 of the body 19. The electro-muscular stimulator 18a positioned in the side 40 (e.g. for positioning over the rear of the joint 9) can be used to receive electrical stimulation signals from the controller 14 for application approximately centrally to the rear of the joint 9. The electro-muscular stimulator 18a positioned in the side 38 of the body 19 (e.g. for positioning over the front of the joint 9) can be used to receive electrical stimulation signals from the controller 14 for application to one side of front of the joint 9, meaning that positioning of the electro-muscular stimulator 18a is asymmetric about the joint in the zone 14. In other words, the electro-muscular stimulator 18a in the side 38 is positioned closer to the position retainer 12 of the end 30 and thus relatively further away from the position retainer 12 of the end 32. One example application of the sleeve 10 is with respect to the knee joint 9, such that the electro-muscular stimulator 18a in the side 38 is for positioning above the knee joint 9 (i.e. between the knee and the hip, such that band 12 adjacent to the end 30 is of greater diameter than the band 12 adjacent to end 32). It is also recognised that the electro-muscular stimulators 18a can be positioned in other areas of the sensor platform 10 (e.g. sleeve or other portion of the sensor platform 10 incorporated in a garment 11 (e.g. underwear such as jockey shorts, bra, etc.), the other area(s) spaced apart from any joints 9 covered by the garment 11.

It is recognized that the electro-nerve stimulators 18a can be positioned in the intermediate zone 14, such that one or both of the electro-nerve stimulators 18a can be present in the zone 14 of the body 19. The electro-nerve stimulator 18a positioned in the side 40 (e.g. for positioning over the rear of the joint 9) can be used to receive electrical stimulation signals from the controller 14 for application approximately centrally to the rear of the joint 9. The electro-nerve stimulator 18a positioned in the side 38 of the body 19 (e.g. for positioning over the front of the joint 9) can be used to receive electrical stimulation signals from the controller 14 for application to one side of front of the joint 9, meaning that positioning of the electro-nerve stimulator 18a is asymmetric about the joint in the zone 14. In other words, the electro-nerve stimulator 18a in the side 38 is positioned closer to the position retainer 12 of the end 30 and thus relatively further away from the position retainer 12 of the end 32. One example application of the sleeve 10 is with respect to the knee joint 9, such that the electro-nerve stimulator 18a in the side 38 is for positioning above the knee joint 9 (i.e. between the knee and the hip, such that band 12 adjacent to the end 30 is of greater diameter than the band 12 adjacent to end 32). It is also recognised that the electro-nerve stimulators 18a can be positioned in other areas of the sensor platform 10 (e.g. sleeve or other portion of the sensor platform 10 incorporated in a garment 11 (e.g. underwear such as jockey shorts, bra, etc.), the other area(s) spaced apart from any joints 9 covered by the garment 11.

Referring again to FIGS. 3,4,5,6,7, temperature sensors 18b in the sides 38,40 are for providing temperature measurement signals (e.g. continuously) to the controller 14. As such, the temperature sensors 18b provide temperature readings of the intermediate zone 14 in the side 38 and of the one or more zones 16 in the side 40, such that side 38 is opposed to side 40. In terms of the configuration of side 40 with respect to sensor 18 placement, the actuator 18a is positioned between the temperature sensors 18b. It is also recognized that the temperature sensors 18b positioned in the side 38 of the body 19 (e.g. for positioning over the front of the joint 9) can be used to measure/collect temperature signals to the controller 14 for application to both/all sides of front of the joint 9, meaning that positioning of the temperature sensors 18b is somewhat symmetric about the joint 9 in the zone 14. In other words, the temperature sensors 18b in the side 38 is positioned both towards to the position retainer 12 of the end 30 and towards the position retainer 12 of the end 32. Preferably, the temperature sensors 18b are positioned adjacent to each of the bands 12.

Referring again to FIGS. 3,4,5,6,7, heat actuator 18c is positioned in the side 38 and spans both the zone 14 and adjacent zone 16 closer to the end 30. The heat actuator 18c for applying (e.g. periodically or continuously) an electrical signal (e.g. as heat) to the skin and underlying muscles of the wearer adjacent/underlying the heat actuator 18c. Further, the heat actuator 18c can be of elongate shape and of asymmetric shape about the joint 9, meaning that a majority of the heat actuator 18b is closer to end 30 than to end 32. It is recognized that the heat actuator 18c can be only positioned in one of the opposed sides 38, 40, e.g. side 38, so as to facilitate active heating (via the heat actuator 18c) on one side 38 of the body 19 while facilitating passive cooling (at the same time as active heating application via the heat actuator 18c) on the opposed side 40 of the body 19.

Referring again to FIGS. 3,4,5,6,7, stretch sensors 18d are positioned in the side 36 and spans both the zone 14 and adjacent zones 16 closer to the ends 30,32. Further, the stretch sensor 18d positioned in the side 38 can be positioned only in the zones 16 and not in the zone 14. For example, the stretch sensor 18d can be inly in the side 36 and absent from the opposed side 34, as desired (for example to help with positioning the sleeve 10 by the wearer on one limb/leg/arm/wrist verses the other). The stretch sensors 18d are for providing one or more electrical signals to the controller 1 to be indicative of the angle of flexion of the joint 9 (e.g. how bent or straight the joint 9 is at any time while the sleeve 10 is being worn by the wearer). Further, the stretch sensors 18d can be of elongate shape and of symmetric or asymmetric shape about the joint 9. For example, the stretch sensors 18d can be used to provide signals to the controller 14 indicative of continuous monitoring of joint 9 flexure and extension. For example, the stretch sensors 18d can be used to provide signals to the controller 14 indicative of continuous monitoring of body 8 swelling or stretching.

As further discussed below, the controller 14 can also contain sensors 18 (e.g. non-textile based sensors) such as but not limited to accelerometers 18 for detecting the movements of the wearer such as but not limited to walking, standing, lying, and sitting—e.g. associated with roll, pitch and yaw movements).

In general, the sensors 18 can include further types such as but not limited to: bio impedance sensors 18 positioned to measure fluid buildup in the body 8 as indication of potential infection; respiration sensors 18 to measure amount of perspiration of the body 8, BIA/GRS (galvanic skin response sensors) to measure skin conductivity; ECG sensors 18 to measure electro cardiograph readings; EMG sensors 18 for measuring electrical activity produced by skeletal muscles; pressure sensors/actuators 18 for measuring or otherwise applying pressure with respect to the body 8; chemical sensors/actuators 18 for measuring or otherwise applying chemicals/medicines with respect to the body 8; EEG sensors 18 as an electrophysiological monitoring method to record electrical activity of the brain; as well as shape shifting/adapting actuators 18 for applying a haptic sensation to the body 8 via changes in the shape/form of the fabric of the body 19 containing the shifting/adapting actuators 18. As such, it is recognized that the sensors/actuators 18 can include both passive and active functionality.

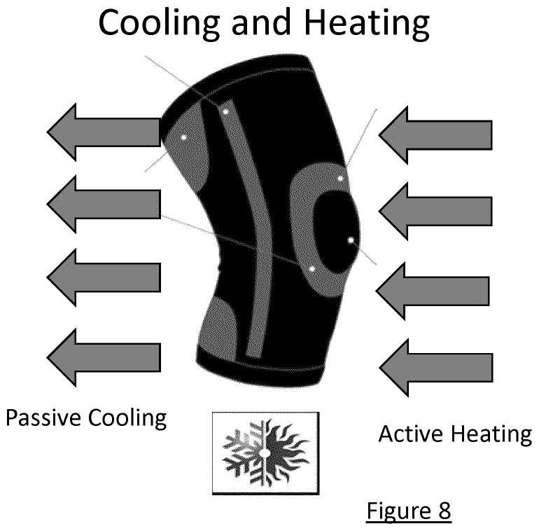

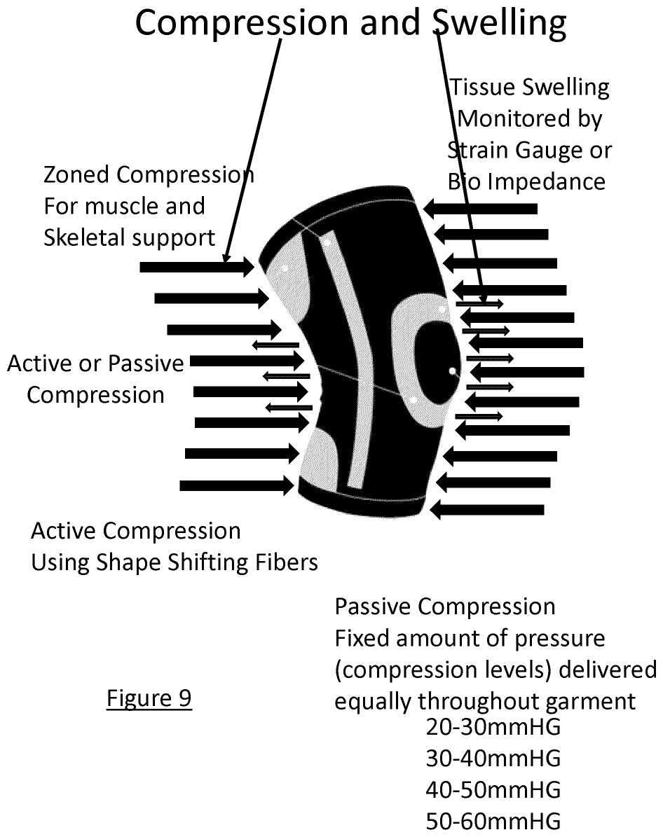

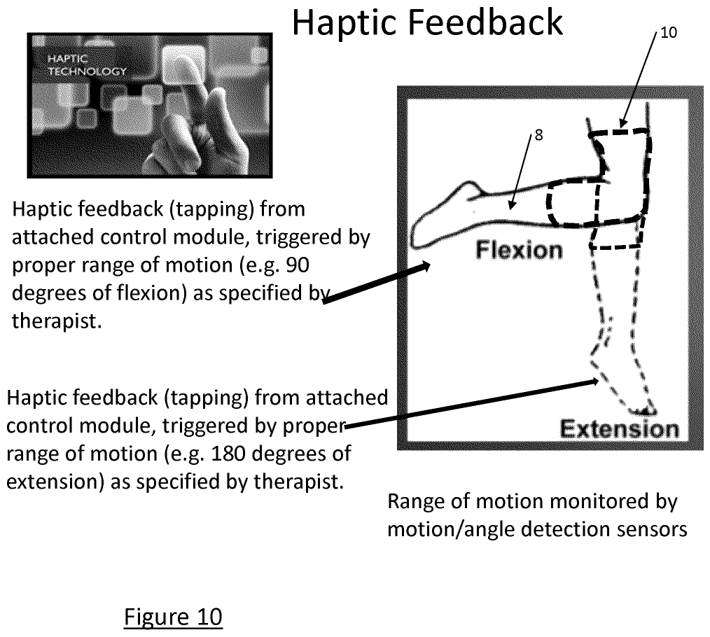

In view of the above, as further discussed below, the sensors/actuators 18 can provide for a plurality of features as applied/measured by the textile computing platform, for example such as but not limited to: heating; cooling; compression/support (e.g. passive/continuous, active/dynamic); monitoring of swelling; monitoring of skin temperature; and/or monitoring of range(s) of motion with haptic feedback provided as desired. For example, FIG. 8 shows an example of the textile computing platform 10 used to provide both active and passive heating/cooling via the proper positioning of the heat actuators 18c and temperature sensors 18b. FIG. 9 shows an example application of sensors/actuators 18 for providing compressive forces via the body 19 (i.e. via the textile shape shifting or otherwise pressure applying actuators 18 in the fabric layer of the body 19) to the body 8 while at the same time measuring the extent of swelling of the body 8 via the body 19 (i.e. via the textile strain sensors 18 in the fabric layer of the body 19). FIG. 10 shows an example of Haptic feedback provided via the series of sensors/actuators 18 via the body 19 to the body 8, for example by applying compressive forces via the body 19 (i.e. via the textile shape shifting or otherwise pressure applying actuators 18 in the fabric layer of the body 19) to the body 8 while at the same time measuring the angle positioning of the body 8/joint 9 via the body 19 (i.e. via the textile strain sensors 18 in the fabric layer of the body 19).

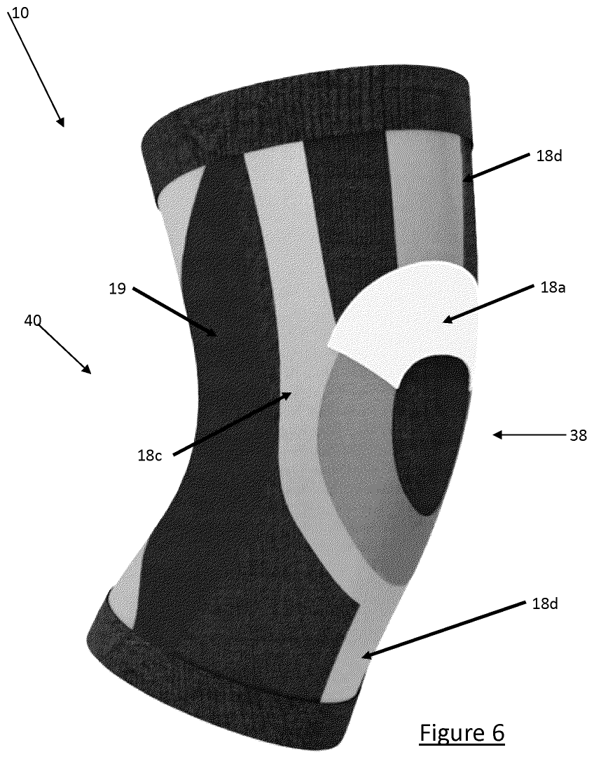

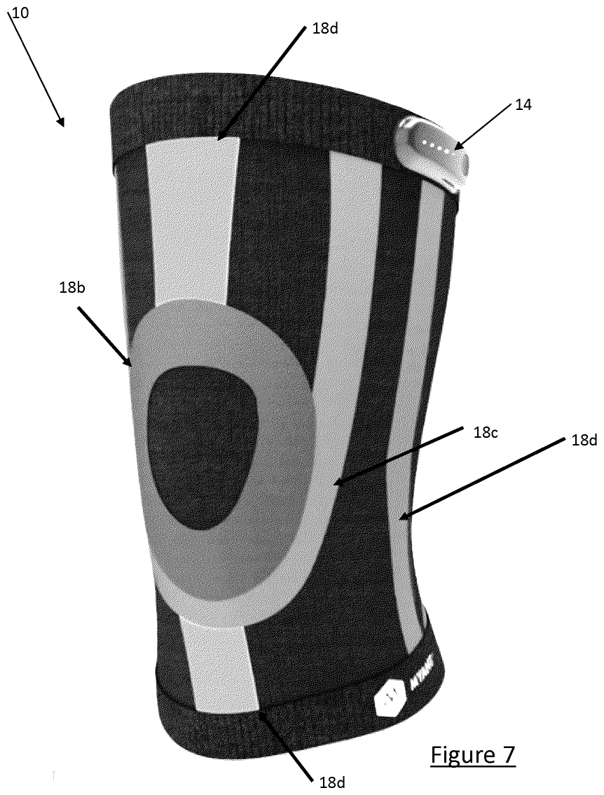

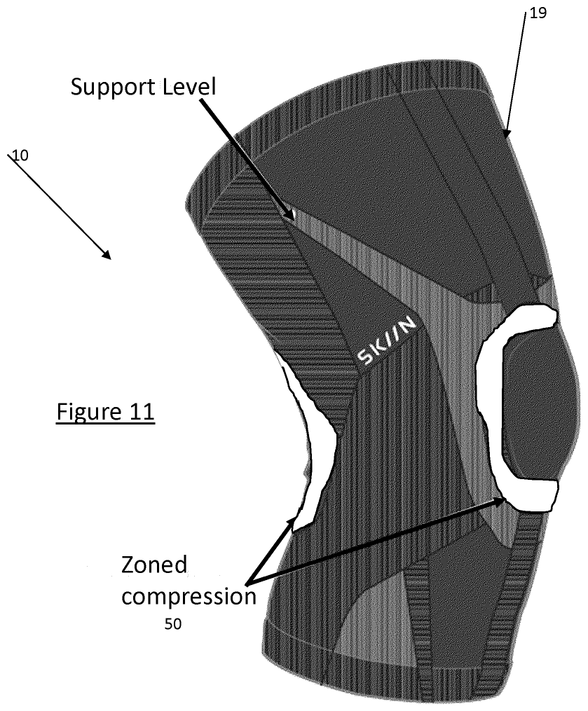

Referring to FIG. 11, shown is an embodiment of the sleeve 10 (e.g. knee brace) having one of more portions of the body 19 configured to apply compressive forces to the body 8 (see FIG. 1). For example, the two portions 50 located on either side 38, 40 (see FIGS. 3-7) can be provided as passive compression (e.g. via passive fibres knitted/woven in a specified pattern such as ribs to induce preferential compression/pressure to those areas of the body underlying the portions 50). Alternatively, or in addition to, the portions 50 could provide active compression (i.e. controlled via electric actuation signals provided by the controller 14) using one or more pressure (e.g. shape shifting fibres) actuators 18 knitted/woven in the fabric layer of the body 19.

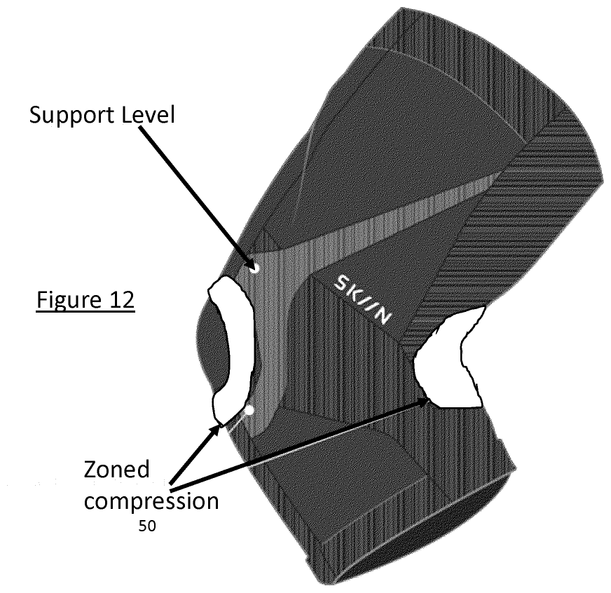

FIG. 12 shows a further embodiment of the sleeve 10 as an elbow brace. As well, the sleeve 10 (e.g. elbow brace) can have one of more portions 50 of the body 19 configured to apply compressive forces to the body 8 (see FIG. 1). For example, the two portions 50 located on either side 38, 40 (see FIGS. 3-7) can be provided as passive compression (e.g. via passive fibres knitted/woven in a specified pattern such as ribs to induce preferential compression/pressure to those areas of the body underlying the portions 50). Alternatively, or in addition to, the portions 50 could provide active compression (i.e. controlled via electric actuation signals provided by the controller 14) using one or more pressure (e.g. shape shifting fibres) actuators 18 knitted/woven in the fabric layer of the body 19.

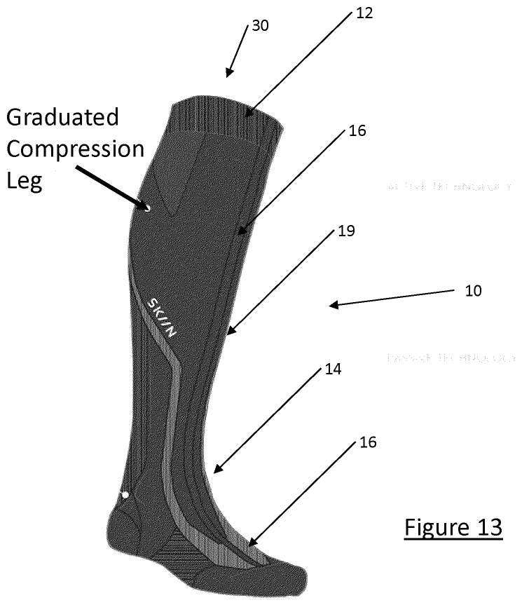

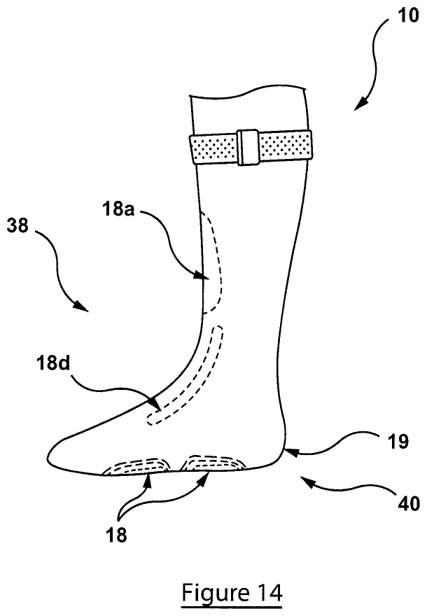

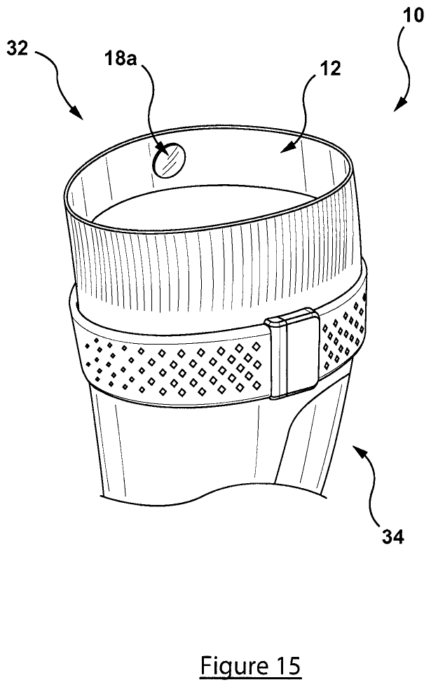

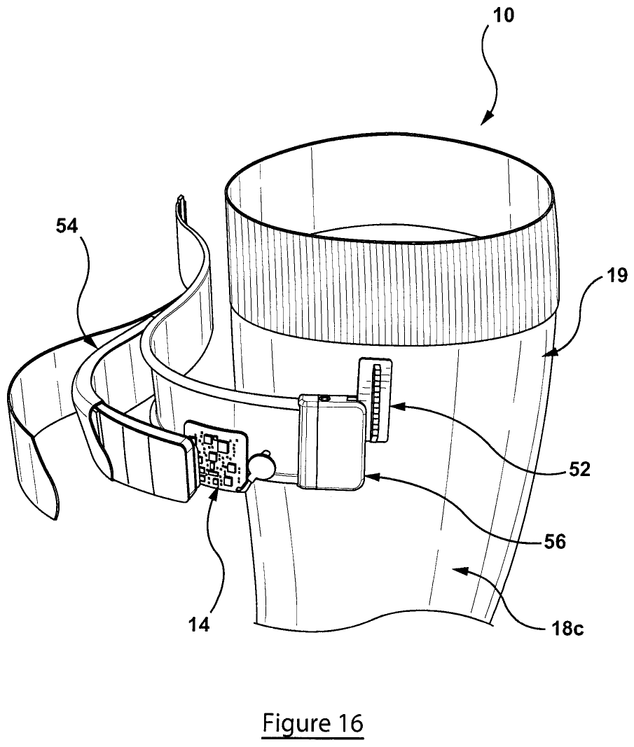

Referring to FIG. 13, shown is a further embodiment of the textile computing platform 10 configured as a sock sleeve 10 having only one band 12 (e.g. for retaining) at one end 30. As provided above, the body 19 has a pair of zones 16 on either side of an intermediate zone 14 (for positioning at the joint 9—e.g. ankle joint). The region 50 of the body 19 can have graduated compression region 50 (e.g. passively applied as per described above), rather than (or in addition to) any retaining properties of the band 12. Referring to FIG. 14, shown are various specific positioning of sensors 18, 18a, 18d in the fabric layer of the body 19, in particular with respect to the zones 14,16 and sides 38,40 as shown. FIG. 15 shows positioning of the actuator 18a in the band 12, on one or more of the sides 32,34. Referring to FIG. 16, shown is an electrical connector 52 mounted on the body 19 and coupled to the electrical circuits 17 (see FIG. 2). As such, the controller 14 can be mounted on a substrate 54 (e.g. a strap) and can have a mating electrical connector 56 for electrically connecting to the electrical connector 52. In this manner, the controller 14 can be used to send and receive electrical signals from the sensors/actuators 18 (see FIG. 14 for example) while the sleeve 10 is being worn by the wearer, while also being versatile to be removed for washing of the sleeve 10 when not in use by the wearer. As noted, the body 19 can have heating elements 18c positioned on any or all of the zones 14,16, as desired.

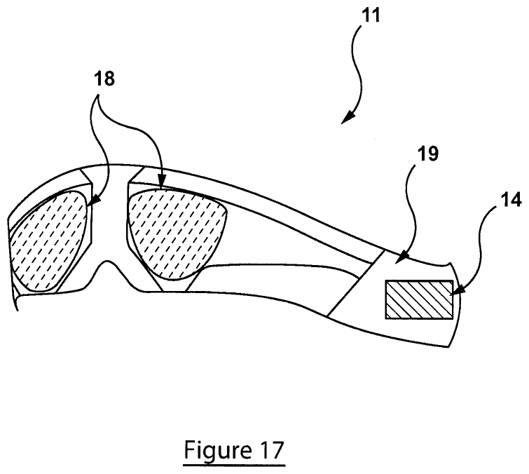

Referring to FIG. 17, shown is another embodiment of the garment 11 (e.g. a sleeve 10) used to be positioned over the wearer's head and eyes, e.g. an eye mask 11. It is recognized that this embodiment does not have an application for positioning with respect to a joint 9 (see FIG. 1), however does have one or more sensor/actuators 18 in the fabric layer of the body 19, as well as the sensors/actuators 18 being coupled electrically to the controller 14. The sensors 18 can be positioned in various areas of the body 19 (the same or other than shown) in order to capture EEG and/or EOG signals, meant to determine sleep stages of the wearer as interpreted by the controller 14 and/or computing device 23. Other applications can include being used for brain machine interface (BMI or BCI). The actuators 18 in the eye mask 11 can be lighting arrays over the eye sockets that can be used to induce lucid dreaming. Alternatively, the actuators 18 can be heating actuators for comfort or bone conduction based audio signal transfer for a calming effect.

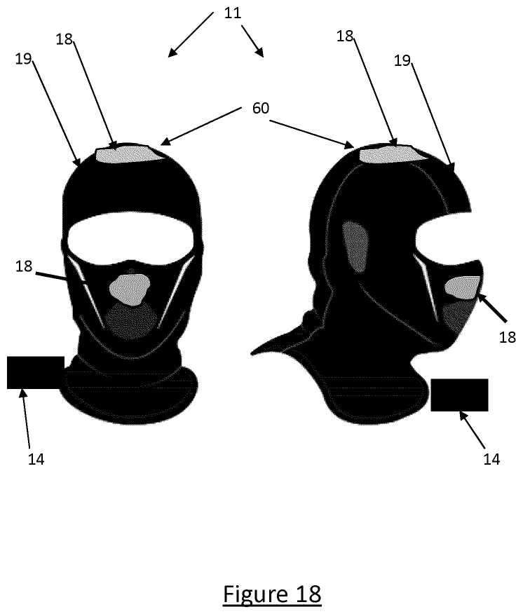

Referring to FIG. 18, shown is another embodiment of the garment 11 (e.g. a sleeve 10) used to be positioned over the wearer's head, e.g. a balaclava 11. It is recognized that this embodiment does not have an application for positioning with respect to a joint 9 (see FIG. 1), however does have one or more sensor/actuators 18 in the fabric layer of the body 19, as well as the sensors/actuators 18 being coupled electrically to the controller 14. The sensors 18 can be positioned in various areas of the body 19 (the same or other than shown) in order to temperature readings (e.g. at a top portion 60 of the head). Further, chemical sensors 18 can be positioned in the mouth/nose area 62 to detect certain agents in the breath of the wearer as interpreted by the controller 14 and/or computing device 23. As well, IMU in the controller 14 and/or stretch sensors 18 positioned in the body 19 can be used to detect movements ultimately for the detection of concussions. It is also recognized that heating actuators 18 can be included in the body 19.

As discussed above, shown are example textile based computing platforms 10, e.g. a fabric sleeve 10, as non-limiting examples of the textile based computing platforms 10 separate to or otherwise integrated into the garment 11, preferable having a resilient knit type, for fitting around a body 8 part of the wearer, in order to collect and receive different modes/types of biometric data based on the type/number of sensors/actuators 18 positioned either on or otherwise knit/woven (e.g. embroidered, interlaced) into the fabric making up the body 19. It is further recognized that the sensors/actuators 18 can be integrated into the fabric (e.g. textile) of the textile based computing platforms 10 in one or more locations of the textile based computing platforms 10, hence providing for a distributed or a localized sensor platform(s) of the textile based computing platforms 10. For example, the textile based computing platform 10 can be a sleeve for fitting over a limb or other extremity (e.g. head, neck, foot, ankle) of the wearer, can be a form fitting article of clothing for fitting over the torso of the wearer, the midsection (including the buttocks) of the wearer and other body 8 parts of the wearer as would be apparent to a person skilled in the art for practicing the invention(s) as claimed herein. Also described, are biometric data collected (i.e. representative of biosignals generated by the body 8 of the wearer). As further described below, the data can be collected from the wearer using the sensors/actuators 18 (e.g. ECG readings, temperature readings, etc.) and can also be applied to the wearer (generating heat, generating vibration, generating pressure, etc. for application to the skin/body of the wearer). It is also recognized that the wearer can generate signals or otherwise interpret data using functionality (e.g. user interface selection(s)) of their device application 23.

Example Sensors 18

It is recognized that selected ones of the sensors/actuators 18 can be unidirectional (i.e. used to collect biometric signals representing the data from the wearer) or bidirectional used to apply signals representing to the wearer). As discussed, functionality of the textile based computing platform 10 with resident sensors/actuators 18 can cover the body 8 part of the wearer such as but not limited to: waist or abdomen; limb such as a leg or arm; torso/trunk; buttocks; foot or ankle; wrist or hand; and/or head. The textile based computing platform 10 can be provided as a stand-alone article or can be combined/combined into an article of clothing such as but not limited to: underwear (such as but not limited to any type of undergarment including jockey shorts, panties, undershirts, and bras); socks, limb bands (e.g. knee band); shirt (e.g. undershirt); etc. The sensors/actuators 18 of the textile based computing platform 10 can be formed as an integral component of the interlacing of the fibres making up the body 19. The fabric of the body 19 can be comprised of interlaced resilient fibres (e.g. stretchable natural and/or synthetic material and/or a combination of stretchable and non-stretchable materials, recognizing that at least some of the fibres comprising the sensors/actuators 18 are electrically conductive, i.e. metallic).

Shape Shifting Alloy Yarn (i.e. fibre) sensor 18 can be based on development on shape memory fine alloy based yarn, in order to control and dictate shape shifting properties of the sensor 18 through an annealing process applied to the yarn individually and/or to the woven/knit sensor 18 (e.g. patch or garment 11 portion thereof) as a whole. The explored annealing process provided improvements to the ductility, reduction in the hardness and made the alloy yarn more malleable for knitting/weaving. Twisting or breading of the annealed alloy fibres with conventional yarns (such as nylon or polyester) can also be done in order to create a multi-filament yarn which can make it easier to employ in knitting structures as the sensors 18. The Alloy Yarn (i.e. fibre) sensor 18 can also be subjected to combination effects of heat annealing and strain annealing in order to provide for functionality of the respective sensor 18 in shape forming/retaining/shifting properties. As such, one example use of the sensor 18 incorporating the alloy fibres is for providing input and/or output of sensory touch/haptics of the wearer, either from or to the wearer via the signals with respect to the controller 14. In parallel, the control of the shape shifting annealed alloys fibres can be done through laser etching, to create a range of shape shifting profiles along a single fibre strand (or combination of strands), as desired. Also, braiding of the shape shifting alloy fibres can create sensor 18 structure which exhibits a stronger (i.e. predefined) contraction/expansion that could lead to greater (i.e. defined) shape shifting on garments 11.

A thermal yarn fibre for the sensors 18 can be a resistive yarn which has the ability to generate/conduct heat via the application of a current (or generation of a current) through the yarn, i.e. as sensory output/input of the wearer/user implemented by the corresponding application of the device 14,23. The resistance profile of the yarn for the sensor 18 can be adjusted such that it can provide a variety of temperature profiles, as selectable. The developed resistive yarns can be wash tested and certified for daily/regular use such that there can be minimal changes in the resistive properties, i.e. resistive property stability, which could otherwise affect the heating profiles and power requirements of the resistive yarn of the sensors 18.

Piezoelectric Yarns for the sensors 18 can be for housing a plurality of sensory properties (e.g. shape shifting, heat, etc.) in a single filament/fibre. For example, utilization of melting yarns in the sensors 18 can serve as an insulation between active segments (e.g. conductive for heat and/or electricity) of the piezoelectric yarn, all extruded as a single filament. For example, it is envisioned that these yarns will give the ability of producing movement through a new medium on textiles, either from or to the wearer via the signals with respect to the controller 14

Electromagnetic Yarns for the sensors 18 can be used to produce haptic feedback through a magnetic field, e.g. as a sensory input or output. For example, through a coil like knit structure of the sensor 18 and the employment of ferro-magnetic yarn/fibres, the sensors/actuators 18 would have the ability to generate vibrational movements either from or to the wearer via the signals.

Electrical Stimulation fibres of the sensors 18 can provide/receive a seamless and pain-inhibited electrical pulse to/from the skin as a new modality of sensation via textiles. The electrical simulation proficient yarn/fibres can be incorporated in garments 11 on desired locations via and operated via a low (i.e. appropriate) current signal administered via the controller 14 and associated data processing system. For example, electrical pulses can be transmitted to the skin, which can invoke a tactile sensation, either from or to the wearer via the signals.

As discussed, the combination of any of the mentioned sensor/actuation 18 modalities can be employed in generation/sending and receipt/processing of the signals using the controller 14. As such, any of shape shifting alloy, thermal yarn, piezoelectric yarn, electro-magnetic yarn, electrical stimulation yarn can be used in the sensors 18.

The sensors 18 can be composed of Electroactive polymers, or EAPs, which are polymers that exhibit a change in size or shape when stimulated by an electric field. EAPS could also exhibit a change in electrical field if stimulated by mechanical deformation. The most common applications of this type of material are in actuators and sensors. A typical characteristic property of an EAP is that they will undergo deformation while sustaining forces. For example, EPDM rubber containing various additives for optimum conductivity, flexibility and ease of fabrication can be used as a sensor 18 material for measuring electrode impedance measured on human skin of the wearer. Further, EAPs may be used to measure ECG as well as measuring deformation (i.e. expansion of the waist and therefore breathing can be inferred from EAPs). ECG can be measured using surface electrodes, textile or polymer, as desired.

These electrodes 18 can be capable of recording biopotential signals such as ECG while for low-amplitude signals such as EEG, as coupled via pathways with an active circuit of the electrical components within the controller 14. The ECG sensors 18 can be used to collect and transmit signals to the computer processor reflective of the heart rate of the wearer. As such, it is recognized that the electrodes as sensors 18 can be composed of conductive yarn/fibres (e.g. knitted, woven, embroidery using conductive fibres—e.g. silver wire/threads) of the body 19, as desired.

In terms of bioelectrical impedance, these sensors 18 and their measurements can be used in analysis (BIA) via the processor and memory instructions for estimating body composition, and in particular body fat. In terms of estimating body fat, BIA actually determines the electrical impedance, or opposition to the flow of an electric current through body tissues of the wearer interposed between the sensors 18, which can then be used to estimate total body water (TBW), which can be used to estimate fat-free body mass and, by difference with body weight, body fat.

In terms of strain sensing, these sensors 18 can be operated as a strain gauge to take advantage of the physical property of electrical conductance and its dependence on the conductor's geometry. When the electrical conductor 18 is stretched within the limits of its elasticity such that it does not break or permanently deform, the sensor 18 will become narrower and longer, changes that increase its electrical resistance end-to-end. Conversely, when the sensor 18 is compressed such that it does not buckle, the sensor 18 will broaden and shorten, changes that decrease its electrical resistance end-to-end. From the measured electrical resistance of the strain gauge, via the power that is administered to the sensors 18 via the computer processor acting on stored instructions of the controller 14, the amount of induced stress can be inferred. For example, a strain gauge 18 arranged as a long, thin conductive fibres in a zig-zag pattern of parallel lines such that a small amount of stress in the direction of the orientation of the parallel lines results in a multiplicatively larger strain measurement over the effective length of the conductor surfaces in the array of conductive lines—and hence a multiplicatively larger change in resistance—than would be observed with a single straight-line conductive wire. In terms of location/structure of the strain gauge 18, the strain gauge can be located. A further embodiment is where the strain gauge 18 is located in a portion, for example in a serpentine arrangement.

In terms of temperature sensor 18, this sensor is used to measure the dynamic body temperature of the wear. For example, the temperature sensor 18 can be a thermistor type sensor, which is a thermally sensitive resistors whose prime function is to exhibit a large, predictable and precise change in electrical resistance when subjected to a corresponding change in body temperature. Examples cam include Negative Temperature Coefficient (NTC) thermistors exhibiting a decrease in electrical resistance when subjected to an increase in body temperature and Positive Temperature Coefficient (PTC) thermistors exhibiting an increase in electrical resistance when subjected to an increase in body temperature. Other temperature sensor types can include thermocouples, resistance thermometers and/or silicon bandgap temperature sensors as desired. It is also recognized that the sensors 18 can include haptic feedback sensors that can be actuated via the computer processor in response to sensed data processed onboard by the processor and/or instructions. Another example of temperature sensors 18 is where thermocouples could be knitted into the band 19 fabric using textile and coupled directly to the body of the wearer through close proximity/contact in order to get more accurate temperature readings.

The controller 14 can be embodied as a computer device including a computer processor, a memory for executing stored instructions for receiving and processing of data obtained from the sensors 18, as well as communicating via a network interface with a network 25 and external computing device 23 (e.g. Wi-Fi, Bluetooth, attached wired cable, etc.) as well as sending and receiving electrical signals from the sensors 18. The processor, memory and network interface can be mounted on a printed circuit board, which is housed in a housing of the controller 14, as attached to the body 19.

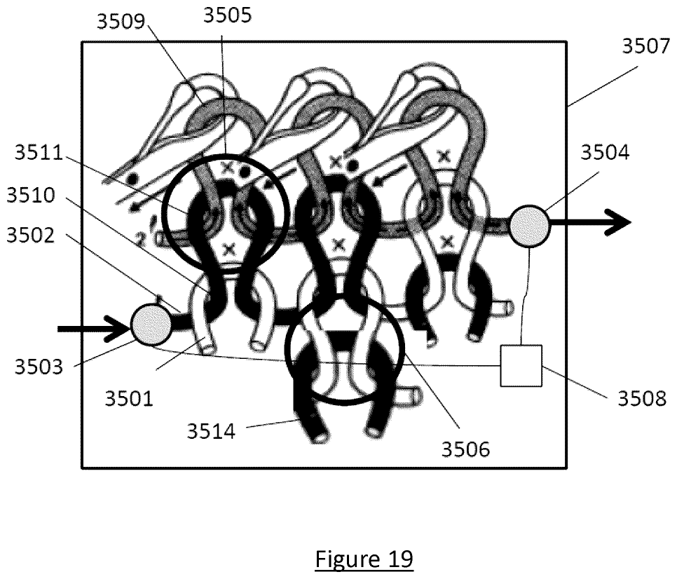

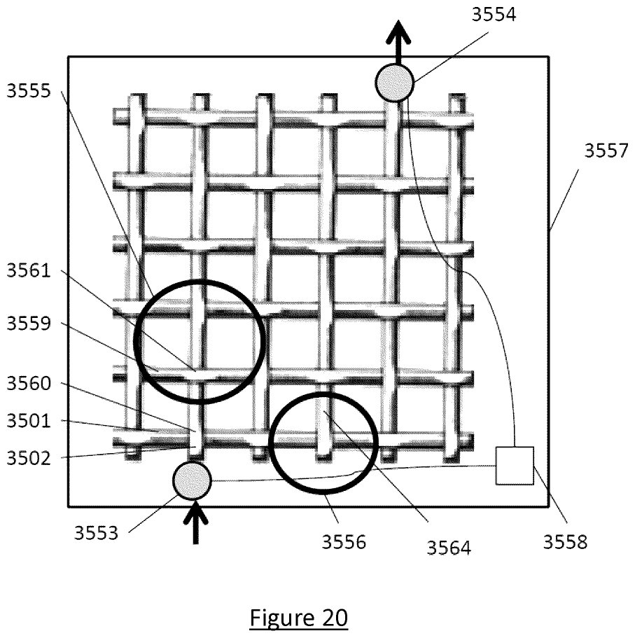

Referring to FIGS. 19 and 20, in one example embodiment, knitting can be used to integrate different sections of the textile (i.e. body 19 fibres incorporating fibres of the sensors/actuators 18) into a common layer (e.g. having conductive pathway(s) and non-conductive sections). Knitting comprises creating multiple loops of fibre or yarn, called stitches, in a line or tube. In this manner, the fibre or yarn in knitted fabrics follows a meandering path (e.g. a course), forming loops above and below the mean path of the yarn. These meandering loops can be easily stretched in different directions. Consecutive rows of loops can be attached using interlocking loops of fibre or yarn. As each row progresses, a newly created loop of fibre or yarn is pulled through one or more loops of fibre or yarn from a prior row. In another example embodiment, can be used to integrate different sections of the textile (i.e. body 19 fibres incorporating fibres of the sensors/actuators 18) into a common layer (e.g. having conductive pathway(s) and non-conductive sections). Weaving is a method of forming a textile in which two distinct sets of yarns or fibres are interlaced at transverse to one another (e.g. right angles) to form a textile.

FIG. 19 shows an exemplary knitted configuration of a network of electrically conductive fibres 3505 in, for example, a segment of an electrically conductive circuit 17 and/or sensor/actuator 18 (see FIG. 1). In this embodiment, an electric signal (e.g. current) is transmitted to conductive fibre 3502 from a power source (not shown) through a first connector 3505, as controlled by a controller 3508 (e.g. controller 14). The electric signal is transmitted along the electric pathway along conductive fibre 3502 past non-conductive fibre 3501 at junction point 3510. The electric signal is not propagated into non-conductive fibre 3501 at junction point 3510 because non-conductive fibre 3501 cannot conduct electricity. Junction point 3510 can refer to any point where adjacent conductive fibres and non-conductive fibres are contacting each other (e.g. touching). In the embodiment shown in FIG. 19, non-conductive fibre 3501 and conductive fibre 3502 are shown as being interlaced by being knitted together. Knitting is only one exemplary embodiment of interlacing adjacent conductive and non-conductive fibres. It should be noted that non-conductive fibres forming non-conductive network 3506 can be interlaced (e.g. by knitting, etc.). Non-conductive network 3506 can comprise non-conductive fibres (e.g. 3501) and conductive fibres (e.g. 3514) where the conductive fibre 3514 is electrically connected to conductive fibres transmitting the electric signal (e.g. 3502).

In the embodiment shown in FIG. 19, the electric signal continues to be transmitted from junction point 3510 along conductive fibre 3502 until it reaches connection point 3511. Here, the electric signal propagates laterally (e.g. transverse) from conductive fibre 3502 into conductive fibre 3509 because conductive fibre 3509 can conduct electricity. Connection point 3511 can refer to any point where adjacent conductive fibres (e.g. 3502 and 3509) are contacting each other (e.g. touching). In the embodiment shown in FIG. 19, conductive fibre 3502 and conductive fibre 3509 are shown as being interlaced by being knitted together. Again, knitting is only one exemplary embodiment of interlacing adjacent conductive fibres. The electric signal continues to be transmitted from connection point 3511 along the electric pathway to connector 3504. At least one fibre of network 3505 is attached to connector 3504 to transmit the electric signal from the electric pathway (e.g. network 3505) to connector 3504. Connector 3504 is connected to a power source (not shown) to complete the electric circuit.

FIG. 20 shows an exemplary woven configuration of a network of electrically conductive fibres 3555. In this embodiment, an electric signal (e.g. current) is transmitted to conductive fibre 3552 from a power source (not shown) through a first connector 3555, as controlled by a controller 3558 (e.g. controller 14). The electric signal is transmitted along the electric pathway along conductive fibre 3552 past non-conductive fibre 3551 at junction point 3560. The electric signal is not propagated into non-conductive fibre 3551 at junction point 3560 because non-conductive fibre 3551 cannot conduct electricity. Junction point 3560 can refer to any point where adjacent conductive fibres and non-conductive fibres are contacting each other (e.g. touching). In the embodiment shown in FIG. 20, non-conductive fibre 3551 and conductive fibre 3502 are shown as being interlaced by being woven together. Weaving is only one exemplary embodiment of interlacing adjacent conductive and non-conductive fibres. It should be noted that non-conductive fibres forming non-conductive network 3556 are also interlaced (e.g. by weaving, etc.). Non-conductive network 3556 can comprise non-conductive fibres (e.g. 3551 and 3564) and can also comprise conductive fibres that are not electrically connected to conductive fibres transmitting the electric signal. The electric signal continues to be transmitted from junction point 3560 along conductive fibre 3502 until it reaches connection point 3561. Here, the electric signal propagates laterally (e.g. transverse) from conductive fibre 3552 into conductive fibre 3559 because conductive fibre 3559 can conduct electricity. Connection point 3561 can refer to any point where adjacent conductive fibres (e.g. 3552 and 3559) are contacting each other (e.g. touching). In the embodiment shown in FIG. 20, conductive fibre 3552 and conductive fibre 3559 are shown as being interlaced by being woven together. Again, weaving is only one exemplary embodiment of interlacing adjacent conductive fibres. The electric signal continues to be transmitted from connection point 3561 along the electric pathway through a plurality of connection points 3561 to connector 3554. At least one conductive fibre of network 3555 is attached to connector 3554 to transmit the electric signal from the electric pathway (e.g. network 3555) to connector 3554. Connector 3554 is connected to a power source (not shown) to complete the electric circuit.

In accordance with one or more of the embodiments, the body 19 layer can be made on a seamless knitting machine where the electrical circuit is an integral part of the textile based computing platform 10, with identical or similar physical properties (stretch, recovery, weight, tensile strength, flex, etc.). The seamless knitting machine can include a circular knit machine manufactured by the SANTONI™ Company, a flat-bed knit machine manufactured by the SHIMA SEIKI® Company, the seamless warp knit machine, and other seamless garment machines, and any equivalent thereof.

In accordance with an embodiment, the knit structure can include a single jersey, a plaited jersey, a terry-plaited jersey, and any equivalent thereof. The plaited jersey can contain nylon or polyester on one side with the SPANDEX™ material covered with nylon or polyester (and any equivalent thereof). The covered SPANDEX™ yarn can be on every feed or on any predetermined pattern or repeat. The nylon or polyester yarn can be of different fineness (denier) ranging from about 10 Denier to about 300 Denier singles or multiple filaments or two-plied or three-plied or any combination and/or permutation as required (and any equivalent thereof) for the final properties of the garment or textile structure. Similarly, the SPANDEX™ material can be selected from about 10 Denier to about 200 Denier and can be covered with nylon or polyester having fineness of about 10 Denier to about 200 Denier (mono-filament and/or multifilament yarns), any combination and/or permutation (and any equivalent thereof) as required for the final properties of the garment or textile structure.

Additionally, the knitted seamless shirt, garment, textile, and any equivalent thereof, can be dyed in atmospheric-dyeing machine (at a temperature of about 212 Fahrenheit) before or after heat setting done with dry heat ranging from about 325 Fahrenheit to about 400 Fahrenheit or by steaming. Other yarns that can be used are cotton, rayon, wool, aramid and others and combination (blend) of one or more (and any equivalent thereof). Various conductive yarns available for use in building and integrating the electrical circuit 17 and/or sensors/actuators 18 into the body layer 19 can be: the X-STATIC® yarns (single-ply, multiple ply, about 50 Denier to about 200 Denier single ply), MAGLON™ yarns (single-ply, two-ply, three-ply), a stainless steel (a mono filament, multi-filaments where the number of filaments can range from about 14 to about 512, and each filament thickness ranging from about 5 microns to about 100 microns), AARCON™ yarns, and other available yarns (such as, copper, indium yarns etc., and any equivalent thereof. The conductive yarns can be combined or bundled to achieve the desired resistive result for developing the sensors/actuators 18 structure in the body 19 layer.

The conductive material can be used as is (bare) or covered with polymer coatings such that the conductive yarns are covered (preferably, fully) in an insulation layer. The insulation can be imparted to conductive yarns with a coating of PVC or any thermoplastic resin (such as, EVA, polyamide, polyurethanes, etc., and any equivalent thereof. The non-conductive yarns (body 19 yarns), which make the remainder (those portions of the body 19 that contain non-conductive fibres that are not segments in the conductive circuit 17/sensors/actuators 18), can be selected from available synthetic fibers and yarns, such as polyester, nylon, polypropylene, etc., and any equivalent thereof), natural fiber and yarns (such as, cotton, wool, etc., and any equivalent thereof), a combination and/or permutation thereof, and each as required for the final properties of the garment or textile structure. The garment body yarns can be wrap or plaited during knitting, wrap in a yarn form (twisted at a number of turns per inch as can be required). The SANTONI® seamless machine can be configured to knit in circular knit (using a desired cylinder size), course after course with capability to generate a plain knit or a pattern knit to enhance the user comfort level of the wearer as well, as adding aesthetic and/or a fashion appearance.

We claim:

1. A textile-based computing platform for wearing by a wearer on both sides of a joint of a body of the wearer, the platform comprising:
a textile body shaped as a sleeve including a first zone for positioning adjacent to the joint, a second zone opposite the first zone for positioned on another side of the joint, and an intermediate zone for positioning over the joint;
a fabric sensor incorporated into a textile layer making up the textile body, the fabric sensor having one or more electrically conductive sensor threads incorporated into the textile layer by at least one of knitting or weaving with other threads making up the textile layer;
a fabric actuator incorporated into the textile layer making up the textile body, the fabric actuator having one or more electrically conductive actuator threads incorporated into the textile layer by at least one of knitting or weaving with the other threads making up the textile layer;
an electrical connector mounted on the textile body for connecting to a controller computing device;
an electronic circuit coupling the electrical connector to the fabric sensor and the fabric actuator, by way of circuit electrically conductive threads connected to the one or more electrically conductive actuator threads and the one or more electrically conductive sensor threads, the circuit electrically conductive threads incorporated into the textile layer by at least one of knitting or weaving with the other threads making up the textile layer;
wherein the controller computing device when connected to the electrical connector bidirectionally communicates electrical signals via the electronic circuit with respect to at least one of the fabric sensor and the fabric actuator.

2. The platform of claim 1; wherein the fabric actuator is provided as a pair of actuators positioned in the first zone and the second zone while being absent from the intermediate zone.

3. The platform of claim 1; wherein the fabric sensor is provided as a pair of sensors positioned in the first zone and the second zone while being absent from the intermediate zone.

4. The platform of claim 1; wherein the fabric sensor is provided as a sensor positioned in the first zone and the intermediate zone while being absent from the second zone.

5. The platform of claim 1; wherein the fabric sensor is provided as a sensor positioned in the first zone, the second zone and the intermediate zone.

6. The platform of claim 1; wherein the fabric actuator is provided as a pair of actuators having a first actuator positioned in the intermediate zone on one side of the joint and a second actuator positioned opposite the first actuator in an opposed section of the intermediate zone.

7. The platform of claim 1; wherein the fabric sensor is selected from the group consisting of: a bio impedance sensor positioned to measure fluid buildup in the body; a respiration sensor to measure amount of perspiration of the body; a BIA/GRS sensor to measure skin conductivity; an ECG sensor to measure electro cardiograph readings; an EMG sensor for measuring electrical activity produced by skeletal muscles; a pressure sensor for measuring pressure with respect to the body; a chemical sensor for measuring chemicals/medicines with respect to the body; and an EEG sensor for electrophysiological monitoring; a temperature sensor for measuring temperature of the body.

8. The platform of claim 1; wherein the fabric actuator is selected from the group consisting of: a shape shifting/adapting actuator for applying a haptic sensation to the body via changes in shape/form of the fabric of the fabric actuator; a pressure actuators for applying pressure with respect to the body; a chemical actuator for applying chemicals/medicines with respect to the body; and a heat actuator for applying heat to the body.

9. The platform of claim 1; wherein the electronic circuit is configured to communicate the electrical signals representing at least one or: heating; cooling; compression; support; swelling; temperature; motion; and haptic feedback.

10. The platform of claim 1; wherein the sleeve is for a knee joint.

11. The platform of claim 1; wherein the sleeve is for an elbow joint.

12. The platform of claim 1; wherein the sleeve is for an ankle joint.
